(19) United States
(12) Patent Application Publication
(10) Pub. No.: US 2025/0256111 A1
(43) Pub. Date: Aug. 14, 2025

(54) NEUROSTIMULATOR SYSTEMS WITH ACCELEROMETER USES

(71) Applicant: Boston Scientific Neuromodulation Corporation, Valencia, CA (US)

(72) Inventors: G. Karl Steinke, Valencia, CA (US); Chirag Shah, Valencia, CA (US); Lisa Denise Moore, Glendale, CA (US)

(21) Appl. No.: 19/033,700

(22) Filed: Jan. 22, 2025

Related U.S. Application Data
(60) Provisional application No. 63/551,991, filed on Feb. 9, 2024.

(51) Int. Cl. A61N 1/372; A61N 1/36

(52) U.S. Cl. CPC A61N 1/37247; A61N 1/3603; A61N 1/36139; A61N 1/37223

(57) ABSTRACT

A system may include a neurostimulator. The neurostimulator may include a waveform generator configured to deliver neurostimulation, a controller operably connected to the waveform generator to control the neurostimulation, and an accelerometer. The controller may be configured to monitor an output of the accelerometer, detect an event from the output of the accelerometer, and perform a neurostimulator action in response to the detected event. The event may include at least one intentional user interaction with the neurostimulator or an unintended neurostimulator event.

### CLAIM OF PRIORITY

This application claims the benefit of U.S. Provisional Application No. 63/551,991, filed on Feb. 9, 2024, which is hereby incorporated by reference in its entirety.

### TECHNICAL FIELD

This document relates generally to medical systems, and more particularly, but not by way of limitation, to systems, devices, and methods for using accelerometer(s) in a medical device.

### BACKGROUND

Medical devices may include devices configured to deliver a therapy to a patient and/or monitors configured to monitor a patient condition via user input and/or sensor(s), For example, these devices may include wearable devices and implantable devices. Some implantable devices may use one or more leads to sense electrical signals or to treat various biological disorders, such as but not limited to spinal cord stimulators (SCS) to treat chronic pain, cortical and Deep Brain Stimulators (DBS) to treat motor and psychological disorders, Peripheral Nerve Stimulation (PNS) including Vagal Nerve Stimulation (VNS), Functional Electrical Stimulation (FES), and other neural stimulators to treat urinary incontinence, sleep apnea, shoulder subluxation, and the like.

Medical devices may be manufactured with components, such as an on-board accelerometer, used to detect force, velocity, acceleration, jerk and the like. Other examples include a resettable shock sensor and an inertial measurement unit (IMU), which may include accelerometers and gyroscopes. The term accelerometer, as used herein, is intended to include components such as resettable shock sensors and IMUs. One example is the device used to provide neural control of central sleep apnea in Mokelke et al (U.S. Pat. No. 8,983,611). As provided in this example, the accelerometer may be used to determine patient posture or to sense respiration.

Current medical devices may require user interaction for certain workflow steps. By way of example, an external trial generator (ETS) may have a button or an implantable pulse generator (IPG) may have a magnet or magnetic sensor which may be used to provide device interaction aside from wireless communication.

### SUMMARY

The present inventors recognize that the accelerometer(s) in a medical device (e.g., neural stimulator) may be used in other applications such as, but not limited to, a user input to stimulators for enhanced workflow operations. For example, various embodiments use an accelerometer of a medical device as a means for interacting with the device for certain workflow steps before and/or after the device is applied to the patient.

An example (e.g., “Example 1”) of a system may include a neurostimulator. The neurostimulator may include a waveform generator configured to deliver neurostimulation, a controller operably connected to the waveform generator to control the neurostimulation, and an accelerometer. The controller may be configured to monitor an output of the accelerometer, detect an event from the output of the accelerometer, and perform a neurostimulator action in response to the detected event. The neurostimulator action may include systems actions such as but not limited to sending commands and making logs for later action. The event may include at least one intentional user interaction with the neurostimulator or an unintended neurostimulator event.

In Example 2, the subject matter of Example 1 may optionally be configured such that the event includes the unintended neurostimulator event and the neurostimulator is external to a patient, the unintended neurostimulator event includes dropping the neurostimulator, and the controller is configured to initiate communication, create a log, or adjust or stop activity in response to dropping the neurostimulator.

In Example 3, the subject matter of any one or more of Examples 1-2 may optionally be configured such that the event includes the unintended neurostimulator event and the neurostimulator is external to a patient, the unintended neurostimulator event includes dropping the neurostimulator, and the controller is configured to estimate at least one of a confidence or a significance of dropping the neurostimulator; and/or estimate at least one of a confidence or significance of an expected patient risk or device harm.

The estimated significance may correspond to a confidence in a true detection and may include a signal magnitude.

In Example 4, the subject matter of Example 1 may optionally be configured such that the event includes the unintended neurostimulator event and the neurostimulator is internal to a patient, the unintended neurostimulator event the patient experiencing an unusual force that has a potential to adversely affect operation of the neurostimulator, and the controller is configured to adjust (e.g., adjusting may include altering or pausing) or stop activity in response to the unusual force.

In Example 5, the subject matter of Example 1 may optionally be configured such that the neurostimulator is external to a patient, the event includes the at least one intentional user action with the neurostimulator, and the at least one intentional user action includes at least one or a combination of two or more of: picking up the neurostimulator, turning over the neurostimulator (e.g., one time or more than one time with a pattern), shaking the neurostimulator back and forth in a direction, moving the neurostimulator in a spatial pattern, moving the neurostimulator in a temporal movement pattern, turning the neurostimulator on one or more axes, tapping the neurostimulator with one tap or a temporal tapping pattern of two or more taps, or using a secondary device to vibrate the stimulator in a temporal vibration pattern. A user that performs the user action may be the patient, a caregiver, members of a clinical care team or medical device support team, or users involved with manufacturing, distributing or transporting the neurostimulator. Examples of combination actions include but are not limited to, combining turning and shaking, tapping and shaking, picking up and turning in one or more axes, picking up and moving in a spatial pattern (within a threshold) and a temporal pattern (within a threshold), and the like.

In Example 6, the subject matter of any one or more of Examples 1 or 5 may optionally be configured such that the neurostimulator is external to a patient, the event includes the at least one intentional user action with the neurostimulator, and the neurostimulator action, performed by the controller in response to the at least one intentional user action with the neurostimulator, includes initiating a communication protocol to create a wireless communication session with the neurostimulator, transitioning to or from a manufacturing mode, transitioning to or from a distribution mode, transitioning to or from a traveling mode, transitioning to or from a trial therapy mode, or transitioning to an implantation mode. This may include the neurostimulator changing an internal state, including activation of components or circuitry, changes in operating system or firmware, and changes in internal power consumption, such as transitioning between low and nominal or operating power states. This may also involve changes in circuitry designed to protect the device, e.g., from falls, temperature or other shocks, battery drain, or other hazards to the device.

In Example 7, the subject matter of Example 6 may optionally be configured such that the initiating the communication protocol includes sending advertising packets in response to the intentional user action.

In Example 8, the subject matter of any one or more of Examples 6-7 may optionally be configured such that the wireless communication session is between the neurostimulator and an external pulse generator (EPG), an external trial stimulator (ETS), an operating room (OR) box, a clinician programmer (CP), a remote control (RC) or a digital application on a phone, tablet or computer.

In Example 9, the subject matter of any one or more of Examples 1, 6, 7 or 8 may optionally be configured such that the neurostimulator is external to a patient, the event includes the at least one intentional user action with the neurostimulator, and the neurostimulator action, performed by the controller in response to the at least one intentional user action with the neurostimulator, includes performing a measurement using the neurostimulator.

In Example 10, the subject matter of any one or more of Examples 1, 6, 7 or 8 may optionally be configured such that the neurostimulator is external to a patient, the event includes the at least one intentional user action with the neurostimulator, and the neurostimulation action is performed during device manufacture, distribution, shipping, in operating room or in clinic.

In Example 11, the subject matter of any one or more of Examples 1, 6, 7, 8, 9 or 10 may optionally be configured such that the neurostimulator is external to a patient, the event includes the at least one intentional user action with the neurostimulator, and the neurostimulator action, performed by the controller in response to the at least one intentional user action with the neurostimulator, includes causing the neurostimulator to turn off, causing the neurostimulator to enter a sleep state or a protective state (e.g., protective to temperature, accelerator, electrical or magnetic, or battery health) or lower-power state, deactivating the accelerometer.

In Example 12, the subject matter of claim 1 may optionally be configured such that the neurostimulator is implanted in a patient, and the event includes the at least one intentional user action with the neurostimulator.

In Example 13, the subject matter of claim 12 may optionally be configured such that the intentional user action includes a tap or one or more taps applied to the patient over the neurostimulator.

In Example 14, the subject matter of any one or more of Examples 12-13 may optionally be configured such that the neurostimulator action, performed by the controller in response to the at least one intentional user action with the neurostimulator, includes delivering a dose of neurostimulation, changing a dose of neurostimulation, switching a neurostimulation program, modifying a neurostimulation program schedule, or changing neurostimulator operating modes.

In Example 15, the subject matter of any one or more of Examples 12-14 may optionally be configured such that the neurostimulator action, performed by the controller in response to the at least one intentional user action with the neurostimulator, includes uploading data from the neurostimulator.

An example (e.g., “Example 16”) includes subject matter (such as a method, means for performing acts, machine readable medium including instructions that when performed by a machine cause the machine to perform acts, or an apparatus to perform). The subject matter may be performed using an accelerometer within a neurostimulator, and may include monitoring an output of the accelerometer, detecting an event from the output of the accelerometer, wherein the event includes an intentional user interaction with the neurostimulator or an unintended neurostimulator event, and performing a neurostimulator action in response to the detected event.

In Example 17, the subject matter of Example 16 may optionally be configured such that the event includes the unintended neurostimulator event and the neurostimulator is external to a patient, and the unintended neurostimulator event includes dropping the neurostimulator.

In Example 18, the subject matter of Example 17 may optionally be configured such that the performing the neurostimulation action in response to the detected event includes initiating communication, creating a log, or adjusting or stopping activity in response to dropping the neurostimulator.

In Example 19, the subject matter of any one or more of Examples 17-18 may optionally be configured such that the performing the neurostimulation action in response to the detected event includes: estimating at least one of a confidence or a significance of dropping the neurostimulator; and/or estimating at least one of a confidence or significance of an expected patient risk or device harm.

In Example 20, the subject matter of Example 16 may optionally be configured such that the event includes the unintended neurostimulator event and the neurostimulator is internal to a patient, and the unintended neurostimulator event the patient experiencing an unusual force that has a potential to adversely affect operation of the neurostimulator or the effects of unaltered operation on the patient, wherein the performing the neurostimulation action in response to the detected event includes adjusting or stopping activity in response to the unusual force. The events may be expected, but undesired, and the stimulator can take appropriate action in response, including adjusting, reducing, changing, pausing, or halting stimulation or therapy. For example, sleeping or driving may be patient activities which drive desired changes to some therapies, and the device may detect those activities and take appropriate action in response.

In Example 21, the subject matter of Example 16 may optionally be configured such that the neurostimulator is external to the patient, and the event includes the intentional user action with the neurostimulator.

In Example 22, the subject matter of Example 21 may optionally be configured such that the intentional user action includes at least one or a combination of two or more of: picking up the neurostimulator, turning over the neurostimulator, shaking the neurostimulator back and forth in a direction, moving the neurostimulator in a spatial pattern, moving the neurostimulator in a temporal pattern, tapping the neurostimulator with one tap or a temporal pattern of two or more taps, or using a secondary device to vibrate the stimulator in a temporal vibration pattern.

In Example 23, the subject matter of any one or more of Examples 21-22 may optionally be configured such that the performing the neurostimulator action in response to the intentional user action with the neurostimulator includes initiating a communication protocol to create a wireless communication session with the neurostimulator, transitioning to or from a manufacturing mode, transitioning to or from a distribution mode, transitioning to or from a traveling mode, transitioning to or from a trial therapy mode, or transitioning to an implantation mode.

In Example 24, the subject matter of Example 23 may optionally be configured such that the neural stimulation action includes the initiating the communication protocol, which includes sending advertising packets in response to the intentional user action.

In Example 25, the subject matter of any one or more of Examples 23-24 may optionally be configured such that the wireless communication session is between the neurostimulator and an external pulse generator (EPG), an external trial stimulator (ETS), an operating room (OR) box or OR generator (ORG), a clinician programmer (CP), a remote control (RC) or a digital application on a phone, tablet or computer.

In Example 26, the subject matter of any one or more of Examples 21-25 may optionally be configured such that the performing the neurostimulator action in response to the intentional user action with the neurostimulator includes performing a measurement using the neurostimulator.

In Example 27, the subject matter of any one or more of Examples 21-26 may optionally be configured such that the neurostimulation action is performed during device manufacture, distribution or shipping.

In Example 28, the subject matter of any one or more of Examples 21-26 may optionally be configured such that the neurostimulation action is performed in operating room or in clinic.

In Example 29, the subject matter of any one or more of Examples 21-28 may optionally be configured such that the performing the neurostimulator action in response to the intentional user action includes causing the neurostimulator to turn off, causing the neurostimulator to enter a sleep state, a protective state, or lower-power state, or deactivating the accelerometer.

In Example 30, the subject matter of Example 16 may optionally be configured such that the neurostimulator is implanted in the patient, and the event includes the intentional user action with the neurostimulator.

In Example 31, the subject matter of Example 30 may optionally be configured such that the intentional user action includes a tap or one or more taps applied to the patient over the neurostimulator.

In Example 32, the subject matter of Example 30 may optionally be configured such that the neurostimulator action includes delivering a dose of neurostimulation, changing a dose of neurostimulation, switching a neurostimulation program, modifying a neurostimulation program schedule, or changing neurostimulator operating modes.

In Example 33, the subject matter of Example 30 may optionally be configured such that the neurostimulator action includes uploading data from the neurostimulator.

An example (e.g., “Example 34”) includes subject matter (such as a non-transitory machine-readable medium including instructions, which when executed by a machine, cause the machine to perform a method performed using an accelerometer within a neurostimulator where the method includes: monitoring an output of the accelerometer; detecting an event from the output of the accelerometer, wherein the event includes an intentional user interaction with the neurostimulator or an unintended neurostimulator event; and performing a neurostimulator action in response to the detected event.

In Example 35, the neurostimulator is external to a patient, the event includes the intentional user action with the neurostimulator, and the intentional user action includes at least one or a combination of two or more of: picking up the neurostimulator, turning over the neurostimulator, shaking the neurostimulator back and forth in a direction, moving the neurostimulator in a spatial pattern, moving the neurostimulator in a temporal pattern, or tapping the neurostimulator with one tap or a temporal pattern of two or more taps.

The present subject matter is not limited to these examples. Another example uses the accelerometer and one or more intentional actions to unlock or modify device characteristics. By way of a non-limiting example, the device may modify a window when a communication session (e.g., BLE (Bluetooth Low Energy)) disconnects, or modify a window when a communication mode is in a higher power and/or lower-latency mode. An example may change the available stimulation parameters (e.g., frequencies, pulse widths, amplitudes, duty cycle, burst duration, and the like). An example may enter, exit, or toggle between entering and exiting a program operation mode such as an auto-adjustment mode or a closed loop mode, a study mode, including ones in which parameters may be sensed and recorded for analysis. An example may affect program sequence by playing, pausing, forwarding, or going back in the sequence. Different intentional actions may be detected by the accelerometer for these different commands. An example may respond to an international action by logging an event and/or alerting a peripheral device about the event, which may be a good, bad or neutral event.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example, an embodiment of a neuromodulation system.

FIG. 2 illustrates, by way of example and not limitation, the neuromodulation system of FIG. 1 implemented in a spinal cord stimulation (SCS) system or a deep brain stimulation (DBS) system.

FIG. 3 illustrates, by way of example and not limitation, an embodiment of a modulation device, such as may be implemented in the neuromodulation system of FIG. 1, that includes sensing circuitry.

FIG. 4 illustrates, by way of example and not limitation, different medical device environments in which various embodiments of the present subject matter use the accelerometer.

FIG. 5 illustrates, by way of example and not limitation, a method performed using an accelerometer within a neurostimulator.

FIG. 6 illustrates, by way of example and not limitation, various types of events that may be detected.

FIG. 7 illustrates, by way of example and not limitation, neurostimulator actions that may be performed in response to one or more of the detected events.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized, and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

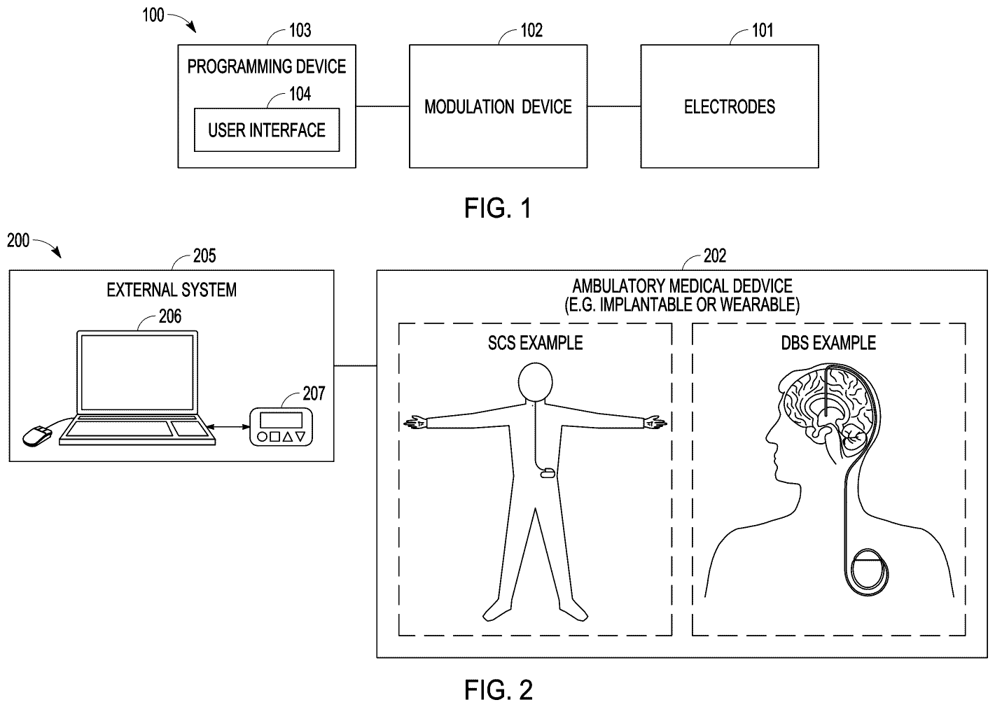

FIG. 1 illustrates, by way of example, an embodiment of a neuromodulation system. The illustrated neuromodulation system 100 includes electrodes 101, a modulation device 102, and a programming system such as a programming device 103. The programming system may include multiple devices. The electrodes 101 are configured to be placed on or near one or more neural targets in a patient. The modulation device 102 is configured to be electrically connected to electrodes 101 and deliver neuromodulation energy, such as in the form of electrical pulses, to the one or more neural targets though electrodes 101. The neuromodulator may also be referred to as a neurostimulator, which is configured to deliver neurostimulation energy. The system may also include sensing circuitry to sense a physiological signal, which may but does not necessarily form a part of modulation device 102. The delivery of the neuromodulation is controlled using a plurality of modulation parameters that may specify the electrical waveform (e.g., pulses or pulse patterns or other waveform shapes) and a selection of electrodes through which the electrical waveform is delivered. In various embodiments, at least some parameters of the plurality of modulation parameters are programmable by a user, such as a physician or other caregiver. The programming device 103 provides the user with accessibility to the user-programmable parameters. The programming device 103 may also provide the use with data indicative of the sensed physiological signal or feature(s) of the sensed physiological signal. In various embodiments, the programming device 103 is configured to be communicatively coupled to modulation device via a wired or wireless link. In various embodiments, the programming device 103 includes a user interface 104 such as a graphical user interface (GUI) that allows the user to set and/or adjust values of the user-programmable modulation parameters. The user interface 104 may also allow the user to view the data indicative of the sensed physiological signal or feature(s) of the sensed physiological signal and may allow the user to interact with that data. The present subject matter is not limited to neural stimulation systems and it may be implemented with medical devices that have an accelerometer such as, but not limited to, medical monitors or cardiac devices such as but not limited to pacemakers and defibrillators.

FIG. 2 illustrates, by way of example and not limitation, the neuromodulation system of FIG. 1 implemented in a spinal cord stimulation (SCS) system or a deep brain stimulation (DBS) system. The present subject matter may be implemented with other neuromodulation systems such as, but not limited to, peripheral nerve stimulation (PNS) devices, functional electrical stimulation (FES) devices, and transcutaneous electrical nerve stimulation (TENS) devices. The illustrated neuromodulation system 200 includes an external system 205 that may include at least one programming device. The illustrated external system 205 may include a clinician programmer 206 configured for use by a clinician to communicate with and program the neuromodulator, and a remote control device 207 configured for use by the patient to communicate with and program the neuromodulator. For example, the remote control device may allow the patient to turn a therapy on and off and/or may allow the patient to adjust patient-programmable parameter(s) of the plurality of modulation parameters. FIG. 2 illustrates a modulation device as an ambulatory medical device 202. Examples of ambulatory devices include wearable or implantable medical devices such as neuromodulators or monitors.

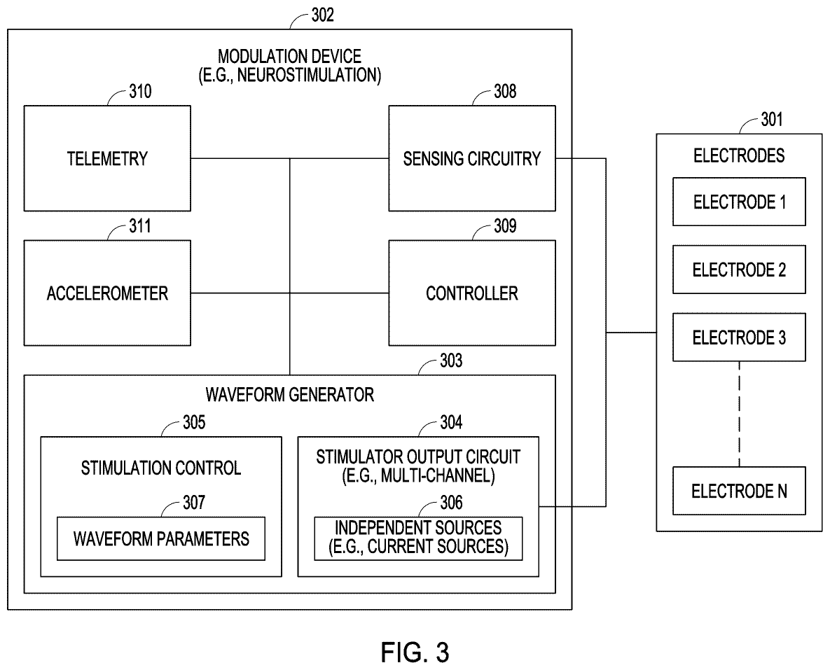

FIG. 3 illustrates, by way of example and not limitation, an embodiment of a modulation device, such as may be implemented in the neuromodulation system of FIG. 1, that includes sensing circuitry. The modulation device 302 (e.g., a neurostimulator) may be configured to be connected to electrode(s) 301, illustrated as N electrodes. Any one or more of the electrodes 301 may be configured for use to deliver modulation energy, sense electrical activity, or both deliver modulation energy and sense electrical activity.

The modulation device 302 may include a waveform generator 303, which may include a stimulator output circuit 304 configured to deliver modulation energy to electrode(s) 301 and a stimulation control 305. The stimulator output circuit 304 may be configured with multiple (e.g., two or more) channels for delivering modulation energy, where each channel may be independently controlled with respect to other channel(s). For example, the stimulator output circuit 304 may have independent sources 306 such as independent current sources or independent voltage sources. The stimulation control 305 may be programmed or otherwise configured with waveform parameters 307, and is configured to control the stimulator output circuit according the waveform parameters 307. By way of example and not limitation, the stimulation control 305 may include start/stop information for the stimulation and/or may include relative timing information between stimulation channels. The stimulation control 305 may include waveform parameters that control the waveform characteristics of the waveform produced by the stimulation output circuit 304. The waveform parameters 307 may include, by way of example and not limitation, amplitude, frequency, and pulse width parameters. The waveform parameters may include, by way of example and not limitation, regular and/or irregular patterns of pulses. The waveform parameters may, but does not necessary, define more than one waveform shape. The stimulation control 305 may be configured to change waveform parameter(s) (e.g., one or more waveform parameters) in response to user input and/or automatically in response to feedback.

The modulation device 302 may include sensing circuitry 308 configured to receive sensed electrical energy from the electrode(s), such as may be used to sense electrical activity in neural tissue or muscle tissue. The sensing circuitry may be configured to process signals in multiple (e.g., two or more) channels. By way of example and not limitation, the sensing circuitry 308 may be configured to amplify and filter the signal(s) in the channel(s). Additionally, or alternatively, the sensing circuitry may be configured for use with many other types physiological sensors.

The modulation device 302 may include a controller 309 operably connected to the waveform generator 303 and the sensing circuitry 308. The controller 309 may be configured to sample a signal produced by the sensing circuitry 308. Examples of features that may be detected include peaks (e.g., minimum and/or maximum peaks including local peaks/inflections), range between minimum/maximum peaks, local minima and/or local maxima, area under the curve (AUC), and curve length between points in the curve. Detected feature(s) may be used as feedback for closed-loop control of the therapy.

The modulation device 302 may include telemetry 310 used to communicate with other devices, such as a device in an external system like a programmer, remote control, or electronic device such as a phone or tablet. The modulation device 302 may include an accelerometer 311. The modulation device 302 may be configured to use the accelerometer to monitor a direction of gravitational pull (e.g., orientation) such as may be indicative of a patient posture, or may use the accelerometer to monitor forces associated with patient motion. The present subject matter implements other functions using the accelerometer.

The accelerometer(s) in a device (e.g., accelerometer 311 in modulation device 302 in FIG. 3) such as a medical device may be used in other applications such as, but not limited to, a user input to the device for enhanced workflow operations. Various embodiments may use an accelerometer of a device as a means for interacting with the device for certain workflow steps. For example, the accelerometer may be used to interact with a neurostimulator before the neurostimulator is implanted or applied to the patient for therapy and/or after the neurostimulator is implanted or applied to the patient for therapy. The neurostimulator may be applied to the patient by having the patient wear the device or by implanting the device in the patient.

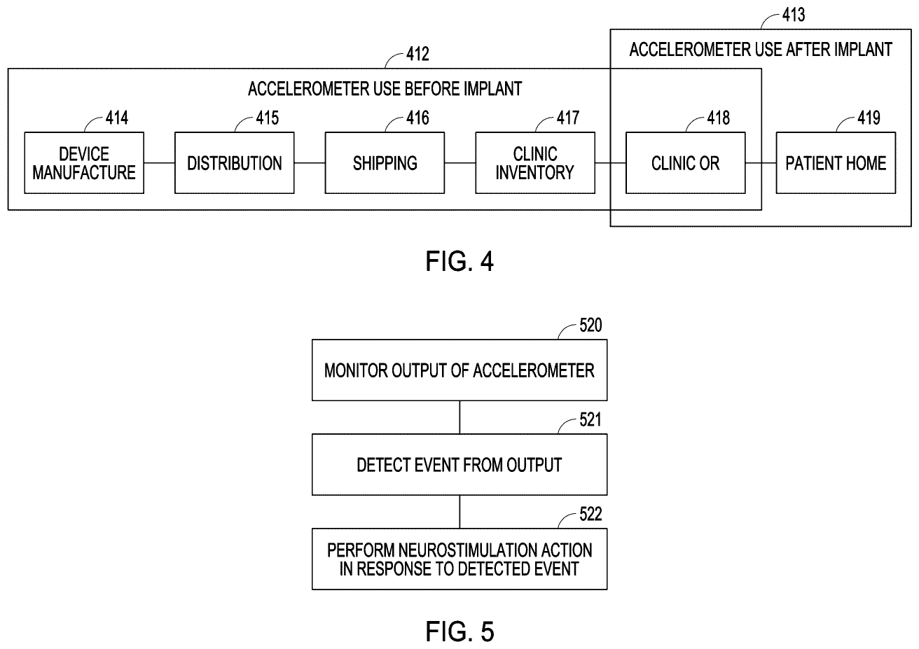

FIG. 4 illustrates, by way of example and not limitation, different medical device environments in which various embodiments of the present subject matter use the accelerometer. In an example in which the medical device is an implantable neural stimulator, the accelerometer may be used before the neurostimulator is implanted 412 and/or may be used after the neurostimulator is implanted 413. Before the neurostimulator is implanted, the neurostimulator may pass through environments or locations where the device is manufactured 414, distributed 415, shipped 416, inventoried for use by a clinic 417 and/or prepped for surgical implantation in a clinic operating room (OR) 418. Examples of workflow steps that may require device interaction prior to the implantation of the device may include pairing the device with another device such as a programmer, phone or computer. After the device is implanted, the accelerometer may be used in the operating room 418 or the patient's home 419. The present subject matter may be implemented by various users during different timepoints of the device. Example users may include a manufacturer, field personnel, clinicians (e.g., implanter such as a neurosurgeon, fellow or surgical staff, or programmer such as a NL, fellow, nurse or staff), patient or patient caregiver. Example timepoints may include various times during manufacture, various times during distribution, various times during shipping, various times during receiving, various times into or within a clinic such as various times at device implant (e.g., trial or permanent device) and/or programming, or various times at home (e.g., trial or permanent device).

A coded pattern of movements, shakes and/or taps may be used to communicate with the neurostimulator using the accelerometer. For example, a user may tap the implanted device to increase or decrease the stimulation and/or to give a dose of neurostimulation. For example, a user may notice an aura for an oncoming seizure and tap the implanted neurostimulator to give a dose of neural stimulation or to implement a neurostimulation program for treating seizures. In an example, a tap or pattern of taps may be used to change modes, such as to enter/exit pairing, or advertising for wireless communication, entering/exiting an MRI mode, pausing/resuming closed-loop stimulation with sensor feedback. In an example, a user may pick up the device to a device state, such as stimulation, sensing, telemetry (strength), battery charge, and the like. A coded pattern may be used to deliver an associated stimulation and/or recording.

There are a number of examples provided herein for using the accelerometer to interact with the device. Depending on the specific interaction, various benefits may include device longevity or battery savings when the device is used to enter and/or exit a power saving (e.g., “sleep” mode) or reduce energy expenditure for wireless communication such as by reducing an advertising interval for BLE (Bluetooth Low Energy), or may include ease of use such as reducing or simplifying workflow steps. For example, a simple movement of the device may be used to initiate pairing of the device with another device. In an operating room (OR), a user may make a predefined motion (e.g., two chops) using the device to cause the device to enter a pairing mode.

Different actions or motions may be detected. In a non-limiting example, a device fall may be detected within an operating room before the device is implanted, and the device may be configured to stop stimulation when the fall has been detected. In another example a user fall may detected. The device, or other part of the system, may be configured to contact an emergency contact or emergency services upon detection of the fall. The device may be configured to adjust stimulation in response to a fall.

Example conditions include packaging an EPG or IPG (e.g., on table), an ETS on the patient's body or an IPG in a patient's body. Example interactions may include picking up, turning over, shaking, figure-eight patterns or other patterns, tapping patterns, and fall estimation.

Example operations may include paring the device with the EPG, ETS, or OR Box by picking ups, tapping or other device interaction. An example operation may include picking up, tapping or other device interaction to confirm the device is the intended device. An example operation may include re-establishing telemetry (or otherwise causing a device in an energy-saving sleep mode to wake-up or go active). An example operation may include taking a measurement, such as but not limited to measuring impedance, by tapping, picking up or other device interaction. An example operation may include a standard sensing sweep performed by a device with an array of sensors. An example operation may include turning the stimulator over or other device interaction to ignore all accelerometer input and/or turn off the device. An example operation may include detecting a fall of the device in an operating room, an stopping all operations in response to the detected fall. For example, the accelerometer processing circuitry may be configured to detect and distinguish falls which may damage the device from other device motion. An example operation may include a tapping pattern or configuration for an implanted device. For example, stimulator data may be uploaded from the stimulator to a clinician programmer, patient remote control, phone or other external device. An example operation is to determine a patient position, such as determine a position consistent with sleep and flagging recordings as potential for sleep. Some device embodiments may implement sleep-specific programs when the determined patient position is consistent with sleep.

FIG. 5 illustrates, by way of example and not limitation, a method performed using an accelerometer within a neurostimulator. Neurostimulators are used herein as a specific device example. Those or ordinary skill in the art will understand that the method may be implemented with other device, including medical devices and non-medical devices. The method may include monitoring an output of the accelerometer 520, detecting an event from the output of the accelerometer 521, and performing a neurostimulator action in response to the detected event 522. The event may include an intentional user interaction with the neurostimulator or an unintended neurostimulator event. Examples of unintended neurostimulator events may include abnormal events or actions that are somewhat foreseeable but the accelerometer is intended to protect against such as patient falls. By way of example, an event signal template may be associated with a given event. The signal output of the accelerometer may be analyzed using the event signal template. The event may be declared when the analysis indicates that the accelerometer signal output is close to the event signal template.

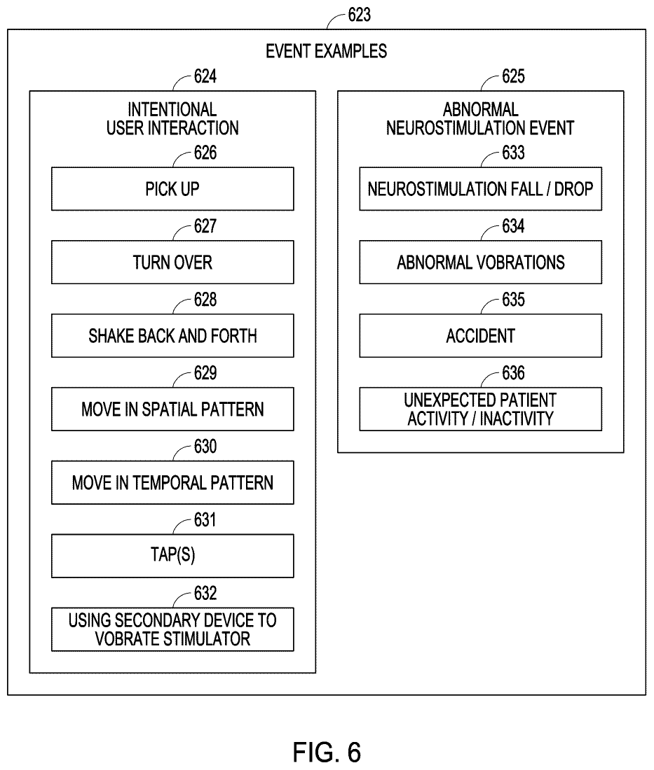

FIG. 6 illustrates, by way of example and not limitation, various types of events that may be detected. The detected events 623 may include intentional user interactions 624 and/or unintended neurostimulator events 625. Examples of intentional user interactions 624 may include picking up the device 626, turning the device over 627, shaking the device back and forth in any pattern or in a predefined path 628, moving the device is a predefined spatial pattern 629, moving the device in a predefined temporal pattern 630, tapping on the device using one tap 631 or a temporal pattern or spatial pattern of taps, or using a secondary device to vibrate the stimulator in a temporal vibration pattern 632. For example, a spatial pattern may include a tap over a subcutaneously-implanted device or may include tap(s) adjacent or more remote to the implanted device. Multiple accelerometers may be used to determine the location of the taps with respect to the implanted device. Examples of unintended neurostimulator events 625 detectable by the neurostimulator may include a fall or a drop of the neurostimulator device 633, abnormal vibrations 634, an accident 635 which may be detected by an unusual force or sequence of forces, and/or an unexpected movement corresponding to an unexpected patient activity and/or unexpected patient inactivity 636. The unintended neurostimulator events 625 may occur when the neurostimulator is implanted or otherwise applied to the patient (e.g., external neurostimulator), or when the neurostimulator is in in manufacturing, distribution, shipping, inventory or in-clinic. The events may be expected, but undesired, and the stimulator can take appropriate action in response, including adjusting, reducing, changing, pausing, or halting stimulation or therapy. For example, sleeping or driving may be undesired patient activities during some therapies, and the device may detect those activities and take appropriate action in response.

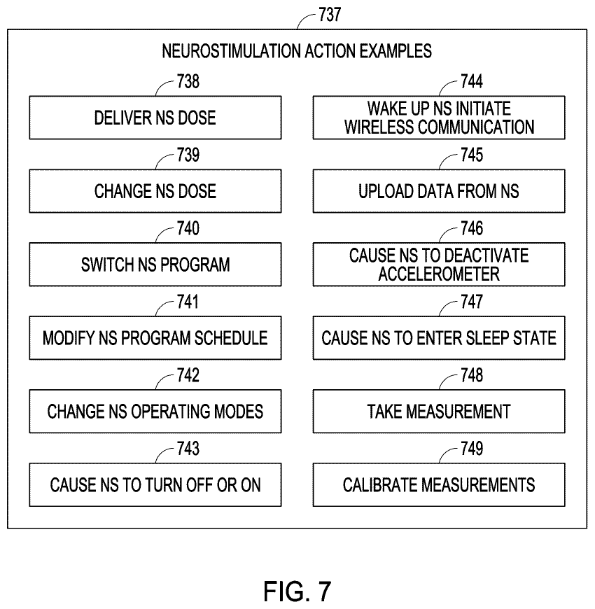

FIG. 7 illustrates, by way of example and not limitation, neurostimulator actions that may be performed in response to one or more of the detected events. The neurostimulator actions 737 may include delivering a dose of neurostimulation 738, changing a dose of neurostimulation 739, switching among neurostimulation programs preprogrammed within the neurostimulator 740, and/or modifying a schedule of neurostimulator programs 741. The schedule may be a preprogrammed sequence and timing for implementing the programs. Additionally, or alternatively, the neurostimulator actions 737 may include changing neurostimulator operating modes 742, causing the neurostimulator to turn on or off 743, waking up the neurostimulator to initiate wireless communication (e.g., advertise) 744, uploading data from the neurostimulator 745, causing the neurostimulator to deactivate the accelerometer 746, causing the neurostimulator to enter a sleep state 747, or causing the neurostimulator to take measurements 748 (e.g., impedance measurements for implanted electrodes), or causing the neurostimulator to calibrate measurements 749. Other neurostimulator actions may be taken. For example, the neurostimulator action may include systems actions such as but not limited to sending commands and making logs for later action. unlock or modify device characteristics. The device may modify a window when BLE disconnects, may change the available stimulation parameters (e.g., frequencies, pulse widths, amplitudes, duty cycle, burst duration, and the like), or may enter, exit, or toggle between entering and exiting a program operation mode such as an auto-adjustment mode or a closed loop mode, a study mode in which parameters may be sensed and recorded for analysis. The neurostimulator action may be an action on a program sequence (e.g., playing, pausing, forwarding, or going back in the sequence). One motion may correspond to one of the commands. Different motions may correspond to different commands. Another neurostimulator action may include logging an event and/or alerting a peripheral device about the event, which may be a good, bad or neutral event. The neurostimulator action may include turning on or off, causing the neurostimulator to enter a sleep state, a protective state, or lower-power state, or deactivating the accelerometer. Other neurostimulator actions may include initiating a communication protocol to create a wireless communication session with the neurostimulator, transitioning to or from a manufacturing mode, transitioning to or from a distribution mode, transitioning to or from a traveling mode, transitioning to or from a trial therapy mode, or transitioning to an implantation mode. These modes may involve transitioning to a protective or lower power state for the device. This may include the neurostimulator changing an internal state, including activation of components or circuitry, changes in operating system or firmware, and changes in internal power consumption, such as transitioning between low and nominal or operating power states. This may also involve changes in circuitry designed to protect the device, e.g., from falls, temperature or other shocks, battery drain, or other hazards to the device. Furthermore, for other medical or non-medical devices, there may be many other device actions that may be triggered by the output of one or more accelerometers.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method performed using an accelerometer within a neurostimulator, the method including:
monitoring an output of the accelerometer;
detecting an event from the output of the accelerometer, wherein the event includes an intentional user interaction with the neurostimulator or an unintended neurostimulator event; and
performing a neurostimulator action in response to the detected event.

2. The method of claim 1, wherein the event includes the unintended neurostimulator event and the neurostimulator is external to a patient, and the unintended neurostimulator event includes dropping the neurostimulator.

3. The method of claim 2, wherein the performing the neurostimulation action in response to the detected event includes initiating communication, creating a log, or adjusting or stopping activity in response to dropping the neurostimulator.

4. The method of claim 2, wherein the performing the neurostimulation action in response to the detected event includes:
estimating at least one of a confidence or a significance of dropping the neurostimulator; and/or
estimating at least one of a confidence or significance of an expected patient risk or device harm.

5. The method of claim 1, wherein the event includes the unintended neurostimulator event and the neurostimulator is internal to a patient, and the unintended neurostimulator event the patient experiencing an unusual force that has a potential to adversely affect operation of the neurostimulator or effects of unaltered operation on the patient, wherein the performing the neurostimulation action in response to the detected event includes adjusting or stopping activity in response to the unusual force.

6. The method of claim 1, wherein the neurostimulator is external to a patient, and the event includes the intentional user action with the neurostimulator.

7. The method of claim 6, wherein the intentional user action includes at least one or a combination of two or more of:
picking up the neurostimulator,
turning over the neurostimulator one or more times,
shaking the neurostimulator in one or more directions,
moving the neurostimulator in a spatial pattern,
moving the neurostimulator in a temporal pattern,
turning the neurostimulator on one or more axes,
tapping the neurostimulator with one tap or a temporal pattern of two or more taps, or
using a secondary device to vibrate the neurostimulator in a temporal vibration pattern.

8. The method of claim 6, wherein the performing the neurostimulator action in response to the intentional user action with the neurostimulator includes:
initiating a communication protocol to create a wireless communication session with the neurostimulator;
transitioning to or from a manufacturing mode;
transitioning to or from a distribution mode;
transitioning to or from a traveling mode;
transitioning to or from a trial therapy mode; or
transitioning to an implantation mode.

9. The method of claim 8, wherein the neurostimulation action includes the initiating the communication protocol, which includes sending advertising packets in response to the intentional user action.

10. The method of claim 8, wherein the wireless communication session is between the neurostimulator and an external pulse generator (EPG), an external trial stimulator (ETS), an operating room (OR) box or OR generator (ORG), a clinician programmer (CP), a remote control (RC) or a digital application on a phone, tablet or computer.

11. The method of claim 6, wherein the performing the neurostimulator action in response to the intentional user action with the neurostimulator includes performing a measurement using the neurostimulator.

12. The method of claim 6, wherein the neurostimulation action is performed during device manufacture, distribution or shipping.

13. The method of claim 6, wherein the neurostimulation action is performed in operating room or in clinic.

14. The method of claim 6, wherein the performing the neurostimulator action in response to the intentional user action includes causing the neurostimulator to turn off, causing the neurostimulator to enter a sleep state, a protective state, or lower-power state, or deactivating the accelerometer.

15. The method of claim 1, wherein the neurostimulator is implanted in a patient, and the event includes the intentional user action with the neurostimulator.

16. The method of claim 15, wherein the intentional user action includes a tap or one or more taps applied to the patient over the neurostimulator.

17. The method of claim 15, wherein the neurostimulator action includes delivering a dose of neurostimulation, changing a dose of neurostimulation, switching a neurostimulation program, modifying a neurostimulation program schedule, or changing neurostimulator operating modes.

18. The method of claim 15, wherein the neurostimulator action includes uploading data from the neurostimulator.

19. A non-transitory machine-readable medium including instructions, which when executed by a machine, cause the machine to perform a method performed using an accelerometer within a neurostimulator, the method including:
monitoring an output of the accelerometer;
detecting an event from the output of the accelerometer, wherein the event includes an intentional user interaction with the neurostimulator or an unintended neurostimulator event; and
performing a neurostimulator action in response to the detected event.

20. A system comprising a neurostimulator, wherein the neurostimulator includes:
a waveform generator configured to deliver neurostimulation;
a controller operably connected to the waveform generator to control the neurostimulation; and
an accelerometer,
wherein the controller is configured to:
monitor an output of the accelerometer;
detect an event from the output of the accelerometer, wherein the event includes at least one intentional user interaction with the neurostimulator or an unintended neurostimulator event; and
perform a neurostimulator action in response to the detected event.
